(19) United States
(12) Patent Application Publication
(10) Pub. No.: US 2006/0184057 A1
(43) Pub. Date: Aug. 17, 2006

(54) A Small, Fast Responding Whole-Body Indirect Calorimeter

(75) Inventor: Craig T. Flanagan, Gainesville, FL (US)

Correspondence Address: DR. CRAIG FLANAGAN, 3600 WINDMEADOWS # 102, GAINESVILLE, FL, 32607, US

(73) Assignee: Craig Thomas Flanagan, Salt Lake City, UT (US)

(21) Appl. No.: 10/904,940

(22) Filed: Dec. 6, 2004

(51) Int. Cl. A61B 5/08

(52) U.S. Cl. 600/531

(57) ABSTRACT

A system for determining resting metabolic rate (RMR) based primarily on oxygen consumption is described. The system consists of an enclosed, sealed chamber within which the patient/subject sits or reclines. The rate of volumetric oxygen change in the system is then monitored to determine the subjects' oxygen consumption rate. The rate of the subjects volumetric oxygen consumption is then converted by the system, using the widely accepted Weir formulation, to calculate RMR. The system is capable of making this calculation in under 10 minutes, making the measurement highly convenient for the subject. The system contains a display mounted on or within the chamber to both serve as a user interface, as well as to serve as a platform for informative programming such as advertisements for weight loss and diet centers and nutrition related products. The system contains a money dispensing system in order that users can pay for the metabolic test. Finally, the system is also capable of obtaining two ancillary parameters, percent body fat and respiratory quotient.

### FIELD OF INVENTION

There is considerable interest in determining resting metabolic rate (RMR) as a tool in the fight against the pandemic of obesity. This invention relates to indirect calorimeters used to determine the RMR and ancillary parameters.

### BACKGROUND ART

This invention consists of a hardware system and an algorithm to determine the basal or resting metabolic rate of an individual. Several systems to determine metabolic rate have been described in the literature. Many systems are handheld, tabletop, or cart configured units (U.S. Pat. Nos. 6,645,158, 6,629,934, 6,620,106, 6,616,615, 6,572,561, 6,475,158, 6,468,222, 6,402,698, 6,309,360, 5,179,958, 5,178,155). Such units have an enclosed or one-way airflow path into which the user or subject breathes using a facemask, a hood or a mouthpiece. On-board sensor systems measure the rate of oxygen consumption as determined from this enclosed gas stream. Such systems are fundamentally different from the system described here in that in our invention, the subject is not required to breathe into a mask, a hood, or a mouthpiece. Such systems have the drawback of generally requiring supervision of the device and the test in order that the test be run.

Other systems are known in the art in which the subject is placed in a small room or whole-body enclosure. Our system is similar to such systems in this respect. Two types of such systems are known in the art, these systems are referred to as whole-body or whole-room indirect calorimeters and whole-body or whole-room direct calorimeters. The latter systems measure heat flux from the body to estimate the RMR (U.S. Pat. Nos. 5,135,311, 5,040,541, 4,386,604). The former systems measure partial pressure changes within the chamber of breathing gases to determine the RMR. Our system shows the greatest similarities to these former (whole-body indirect calorimetry) systems. Specifically, our system monitors (primarily) the change in the partial pressure of oxygen within the chamber to determine RMR. Such systems have the significant drawback of being quite large and therefore impractical. Our system differs from the existing systems known in the art, however, in several important ways, primarily in the size of the machine and short time duration of the RMR test. All such differences will be discussed in detail later in this patent. What follows is a description of the mathematical algorithm necessary to construct a small, fast responding whole-body indirect calorimeter.

### DETAILED DESCRIPTION OF THE PREFERRED EMBODIMENT

This patent submission covers the invention in two principle sections. Both sections contain novel art worthy of patent protection. The first section describes the algorithm or mathematical methodology to be used in the invention. The second section describes the hardware required to implement such a system. The invention, as disclosed here, represents the first gas-mediated, small and fast whole-body indirect calorimeter. The invention has novelty in the mathematical techniques employed to miniaturize whole-room indirect calorimeters, in the mathematical techniques employed to obtain the resting metabolic rate parameter, the respiratory quotient parameter, the percent body fat parameter, and in the pneumatic and sensor-based implementation of the system.

### Algorithm

Section 1.

This indirect calorimeter is a closed chamber system in which a small amount of breathing gas is added to the system and a small amount of gas is allowed to escape from the system. The subject is housed inside the chamber, and by breathing, changes the concentration of gases within the chamber (i.e. ppCO₂ and ppO₂). Our system watches the volumetric rate of change of oxygen inside the chamber. Necessary corrections for the ideal gas law (PV=nRT) in the determination of volumetric oxygen rate of change must be taken into account in order that oxygen consumption can be referenced to a known and accepted standard. In this case, corrections for ambient temperature, humidity, and ambient pressure are made to reference the prevailing conditions to STPD (standard temperature and pressure, dry) conditions. STPD conditions assume a temperature of 0° C., a barometric pressure of 1 atmosphere (760 mmHg), and relative humidity of 0% (absolute humidity of 0 mmHg). A symbol reference guide is provided below for ease in understanding the algorithm which follows:

Fₓ Fraction of gas “x” in dry air (unitless).

F_{x,wet} Fraction of gas “x” in wet air (unitless).

νₓ Flow of gas “x” (L/min)

P Ambient (prevailing) barometric pressure (mmHg).

P_{STPD} STPD barometric pressure (760 mmHg).

T Ambient (prevailing) temperature in chamber (° C.)

T_{STPD} STPD temperature (0° C.)

γ STPD conversion multiplier

Vₓ Volume of gas X at ambient (prevailing) conditions (L)

V_{x,STPD} Volume of gas X at STPD conditions (L)

Δₓ Rate of gas “x” consumption by subject (L/min)

V Gaseous Volume of Calorimeter (L)

In order to convert the volumetric representation of the gas to the STPD reference standard, corrections for temperature, barometric pressure, and water vapor must be made. A corollary of the ideal gas law, Charles' law, states that increasing the temperature of a gas proportionately increases the volume of the gas. Another corollary, Boyle's law, states that the volume of a gas varies inversely with the barometric pressure. Finally, the volume of a gas depends on its water vapor content. Combining these three concepts leads to a rather standard formulation for the correction of measured volumetric gas to an STPD standard volume:
γ=(T_{STPD}/T)(P/P_{STPD})(1−F_{X,WET}/F_{X}) (1)

Which leads to the following conversion:
V_{x,STPD}=Vₓ*γ (2)

Having established this routine conversion methodology, I would like to note: All STPD conversions should be made to algorithm variables prior to insertion of the variables into the algorithm. In addition to being “just good form”, such preliminary conversions allow for the STPD conversions to be eliminated from the body of the algorithm and ensure that the derivation of the algorithm, which follows, is significantly less cluttered and easier to follow. Therefore, the algorithm, as derived here, will assume all gases exist at STPD conditions.

We now turn our attention to the overall algorithm purpose. The purpose of the following algorithm is to determine the rate of volumetric consumption of oxygen within the chamber by the subject in a short time span of 60 minutes or less and in a small chamber of 5000 liters or less. The algorithm is designed to maximize system accuracy while minimizing system hardware complexity. It consists of three steps for which the sequence of the first two steps is reversible and inconsequential. The first two steps involve the solution of three equations with three unknowns. The first step in the algorithm takes the gaseous volume of the chamber/subject system, and uses this information in combination with input for various sensor systems to obtain an equation relating the volumetric rate of oxygen consumption by the subject to the volume of the chamber/subject system. The second step consists of the solution of two simultaneous non-linear equations (after substitution in of the equation from step 1) to obtain the gaseous volume V of the chamber with a subject inside. Finally, the third step in the algorithm is merely a conversion of volumetric oxygen consumption to resting metabolic rate (RMR), this being the parameter which ultimately interests us. In addition to RMR, additional parameters of clinical interest, the respiratory quotient (RQ) and percent body fat can be teased out of the data. Inclusion of the methods of calculation of these additional parameters are included in the following algorithm section.

Algorithm Step 1.

The purpose of this portion of the algorithm is to determine the gaseous volume of the chamber with a subject inside. In order to do this, we must set up the basic oxygen consumption equations for the subject/chamber system. Therefore, it can be stated that the volumetric rate of oxygen change within the chamber is equivalent to the volumetric rate of oxygen added to the chamber, minus the volumetric rate of oxygen consumed by the subject, minus the volumetric rate of oxygen leaving the chamber. This is a simple formulation and can be described using differential calculus as follows:
dQ/dt=>{Rate of oxygen flowing into chamber}−{Rate of oxygen leaving chamber} (3)

In the case in which there is no flow going into the chamber and no flow exiting the chamber, which we will now refer to as “flow regime 1”, the equation is quite simple:
dQ/dt=−Δ_{O2}(t) (4)

Where Δ_{O2}(t) represents the volumetric consumption of oxygen by the subject inside the chamber. A simple solution of this linear first order differential equation can be obtained by separation of variables:
dQ=−Δ_{O2}(t)*dt (5)

Integrating produces:
Q(t)=−Δ_{O2}(t)*t+C (6)

Where C is a constant of integration. Now, let's solve for C by proposing the following initial condition:
Q(0)=F_{O2o}(0)*V (7)

Where F_{O2o}(0) is the fraction of oxygen leaving the chamber at the very beginning of the test (represented as “First Sample” in FIG. 1) and V represents the gaseous volume of the chamber. Now plugging this initial condition into Equation 6 and solving for C yields:
Q(0)=−Δ_{O2}(0)*0+C (8)
C=Q(0)=F_{O2o}(0)*V (9)

Next, plugging C into Equation 6 yields the following:
Q(t)=−Δ_{O2}(t)*t+F_{O2o}(0)*V (10)

We know that Q(t) is the equivalent of F_{O2o}(t)*V, therefore we have the following:
F_{O2o}(t)*V=−Δ_{O2}(t)*t+F_{O2o}(0)*V (11)

This is a valuable equation, since in practice, all of the variables can be measured by sensor systems with the exception of V and Δ_{O2}(t). It turns out that the value of Δ_{O2}(t) is an “unknown” in the non-linear equations which are described in the next section of this patent, so lets solve the above equation in terms of Δ_{O2}(t):
Δ_{O2}(t)*t=F_{O2o}(0)*V−F_{O2o}(t)*V (12)
Δ_{O2}(t)=(F_{O2o}(0)*V−F_{O2o}(t)*V)/t (13)

A good selection for t, in order to obtain a well-defined Equation 13, is shown in FIG. 1 as “Second Sample”. Equation 13 will be substituted into the two non-linear equations in the next section. The next section will then produce a solution for two additional unknowns, V and C, and the value of V can be substituted back into equation 13 to obtain a final solution for Δ_{O2}(t) which is our ultimate objective. FIG. 1 shows the result of the above linear decay equation as seen in flow regime 1 using typical values of pod size and patient oxygen consumption.

Algorithm Step 2.

In this section, we set up and solve two non-linear equations. The solution is a unique solution which produces values of C and V (these variables will be explained later). So let's proceed to set up these equations. As mentioned earlier, we know that the volumetric rate of oxygen change within the chamber is equivalent to the volumetric rate of oxygen added to the chamber, minus the volumetric rate of oxygen consumed by the subject, minus the volumetric rate of oxygen leaving the chamber. This simple formulation can be described using differential calculus as follows:
dQ/dt=>{Rate of oxygen flowing into chamber}−{Rate of oxygen leaving chamber} (14)

Where Q represents the volume of oxygen in the chamber. We are now using what we will call “flow regime 2” in which a predetermined amount of flow is entering (and exiting) the chamber. Now, the rate of oxygen flowing into the chamber is simply equal to the flow of ambient gas into the chamber at time t (νᵢ(t)) multiplied by the fraction of oxygen in this entering gas at time t (F_{O2,i}(t)) or:
{Rate of oxygen flowing into chamber}=νᵢ(t)*F_{O2,i}(t) (15)

Next, the rate of oxygen leaving the chamber is the sum of the oxygen consumed by the subject at time t (Δ_{O2}(t)) and the oxygen leaving the chamber by outflow at time t (F_{O2,o}(t)*ν_{O}(t)) or:
{Rate of oxygen leaving chamber}=Δ_{O2}(t)+(F_{O2,o}(t)*ν_{O}(t)) (16)

Since the amount of oxygen in the chamber at time t (Q(t)) is equivalent to the fraction of oxygen leaving the chamber at time t (F_{O2,o}(t)) multiplied by the volume of gas in the chamber (V) we have:
{Rate of oxygen leaving chamber}=Δ_{O2}(t)+(Q(t)/V)*ν_{O}(t) (17)

Now, putting this all together in the form of a first order linear differential equation results in the following:
dQ/dt=(νᵢ(t)F_{O2i}(t))−(Δ_{O2}(t)+(Q(t)/V) (νₒ(t))) (18)

First order linear differential equations can be solved by substitution or alternately, by transformation into the frequency domain using Laplace Transforms and transforming back into the time domain (after algebraic manipulation in the frequency domain) using the inverse Laplace Transforms or, alternately still by separation of variables. These techniques are considered routine mathematics, having said that, alternate mathematical approaches to solving the problem as stated should not be considered novel art. We will solve the first order linear differential equation using the technique of substitution. The general form of a first order linear differential equation is:
dy/dt=p(t)y=r(t) (19)

Where the integrating factor used for substitution is:
e^{∫}^{_{p(t)dt}} (20)

With a general solution of the form:
y=e⁻^{^{∫}}^{p(t)dt}∫[r(t)e^{∫}^{_{p(t)dt}}dt+C) (21)

Where p(t) and r(t) are either constants, or functions of t alone, and C is the so called constant of integration. Therefore, in our case, with a bit of rearrangement of Equation 18, we have:
r(t)=νᵢ(t)F_{O2i}(t)−Δ_{O2}(t) (22)
p(t)=νₒ(t)/V (23)
so:
Q(t)=e⁻⁽^{^{ν}}^{o(t)/V)dt}∫(νᵢ(t)F_{O2i}(t)−Δ_{O2}(t)e⁽^{^{ν}}^{o(t)/V)dt}+C) (24)

Multiplication and integration of terms produces:
Q(t)=(νᵢ(t)F_{O2i}(t)−Δ_{O2}(t))/(νₒ(t)/V)+C e⁻⁽^{^{ν}}^{o(t)/V)t} (25)

Now, in order to solve this equation we need the constant of integration C. The best way to find this constant C is to have an initial condition for the volume of oxygen in the chamber such as Q(0)=450 (Liters). Unfortunately, in order to know the amount of oxygen in the chamber, one must know both the partial pressure of oxygen inside the chamber and the size of the gaseous volume of the chamber with the subject inside the chamber. Since we do not know the size of the subject we do not know the latter. Thus, we need a second equation with the same unknowns, namely V and C, which will give us two equations with two unknowns and therefore a unique solution for both V and C. The reader should be reminded that the value of Δ_{O2}(t) which is, in reality, a third unknown can be substituted in from Equation 13. Equation 13 states Δ_{O2}(t) in terms of V, so when Equation 13 is substituted in, we are back to two unknowns. It should be noted that the time indices in Equation 13 represent the time elapsed in flow regime 1, while the time indices in Equation 25 represent the time elapsed in flow regime 2. To ensure clarity, we will italicize the indices for flow regime 1. Thus, we have the following equation:
Q(t)=(νᵢ(t)F_{O2i}(t)−( (F_{O2o}(0)*V−F_{O2o}(t)*V)/t))/(νₒ(t)/V)+C e⁻⁽^{^{ν}}^{o(t)/V)t} (26)

Two Equations and Two Unknowns

Equation 26 has two unknowns, V and C. All other variables in the equation can be obtained from sensor systems using appropriate sampling techniques. FIG. 1. shows two candidate sample points designated “Third Sample” and “Fourth Sample”. Given that these sensor variables are obtained for two points in time in flow regime 2, we then have two equations with two unknowns describing V in terms of C. Inspection of Equation 26 leads to the conclusion that the equation is not linear in terms of the unknown variables V and C. Thus, standard linear algebra matrix-based techniques for solution of these two equations will not work. It should also be noted that the two equations must be obtained during flow regime 2 with the flow into, and out of the chamber being held constant. Otherwise, the value of C will change and the solution will be flawed.

FIG. 1. shows a typical exponential decay of volumetric oxygen within the chamber with 120 LPM of air flow into the chamber, 120 LPM of gas flow out of the chamber, and 0.2 LPM of oxygen consumption within the chamber. This figure shows a typical non-linear oxygen volume decay profile within the chamber in flow regime 2. As mentioned, two points are selected on this decay curve to obtain the two equations, in terms of V and C, we seek.

A number of approaches might be used to solve this system of two equations, however, since this algorithm is intended for a microprocessor-based system, using a simple numerical substitution method would be a simple strategy. A suggested method of solution then would be substitution of values of V into the two equations while monitoring the difference in the values of C obtained for the two equations. The value of V is then modified until the difference in the values of C is almost zero (within some acceptable threshold of zero).

Plotting the two equations obtained from Equation 26 produces the two non-linear plots shown in FIG. 2. It should be noted that, in general, more error tolerant solutions to variance in equation variables will be obtained with increasing orthonormality at the intersection of the plots. Further, since this algorithm is intended for a sensor-based system in which some degree of noise and non-linear behavior is to be expected from the sensors, a solution which is error-tolerant to sensor input variability is highly desirable. Optimal system configurations should be selected with this normality consideration in mind.

Having numerically solved the two equations resulting from Equation 26, for V, we can now substitute the value of V into Equation 13 to solve for Δ_{O2}(t) or subject oxygen consumption over time which was our goal.

Ancillary Parameters

In addition, having solved for V from the two equations resulting from Equation 26 and Δ_{O2}(t) in Equation 13, we can determine an additional parameter which might be of interest, namely the percent body fat of the subject. This parameter can be calculated as follows:
Percent Body Fat=495/Density−450 (27)

Siri, WE (Body volume measurement by gas dilution. Techniques for Measuring Body Composition, J Broze and A Henschel. Washington D.C.: National Academy of Sciences/National Research Council, 1961, pages 108-17).

Density represents the mass per unit volume of the human body. The mass can be determined easily enough by weighing the individual, or alternately, by building a load cell underneath the pod and taring the load cell for the mass of the pod. The result would be the mass of the subject. What remains to be determined is the volume occupied by the subject's body, which is simply V-Vpod (where Vpod represents the known gaseous volume of the pod without a subject inside). Life Measurement Inc. of Concorde Calif., uses a similar technique (U.S. Pat. No. 05,105,825) based on air displacement within a chamber (or intra-pod pressure change) to determine body volume as a means to assess percent body fat. Such a technique is seen as an accurate alternative to hydrostatic weighing, dual energy X-Ray absorptiometry (DEXA), skin fold calipers, and bioelectric impedance analysis. Since our technique produces an equivalent result, namely the volume occupied by the subject's body, our invention will also be effective in the determination of percent body fat.

Finally, carbon dioxide has not been mentioned as of yet in this patent. It is known in the art that ignoring carbon dioxide production and simply assuming it is equal to 80% of oxygen consumption will lead only to relatively small errors in the determination of resting metabolic rate (RMR) using the Weir Equation (see next section). For this reason and because carbon dioxide sensors add significant cost to indirect calorimetry systems, carbon dioxide is frequently not measured in such systems. Indeed, it can be ignored in this system with little consequence to the ultimate accuracy of the RMR number calculated. However, carbon dioxide measurement is not without its benefits. Indeed, when carbon dioxide levels are monitored, a valuable clinical parameter called the Respiratory Quotient (RQ) can be obtained. The RQ is simply the volumetric production of carbon dioxide divided by the volumetric consumption of oxygen or:
RQ=Δ_{CO2}(t)/Δ_{O2}(t) (28)

RQ can obtained with reasonable accuracy by simply watching the change in partial pressure of oxygen and carbon dioxide over a reasonable time period in flow regime 1. Namely:
RQ≈( (ₚₚCO₂(t)−ₚₚCO₂(0)/(ₚₚO₂(0)−ₚₚO₂(t)) (29)

RQ is of value in determining the proportionate amount of protein, carbohydrate and fat being burned by the subject. Specifically, high RQs (approaching 1) indicate a proportionately large amount of carbohydrate is being burned in the subject's body at the time of the test, while a low RQ (around 0.7) would indicate the subject is burning proportionately large amounts of fat (protein burn rate is quite small relative to fat and carbohydrate and is estimated). This result is due to the stoichiometric quantities of carbon dioxide produced and oxygen consumed during the catabolic breakdown of these distinctly different molecular compounds. It is known in the art that one of the problems with the measurement of carbon dioxide is that while oxygen consumption in subjects tends to stabilize after a minute or two, leading to the accurate determination of oxygen consumption in a short test such as what is described here, the subject's production of carbon dioxide tends to take far longer to stabilize. This is due to the large pool of carbon dioxide or carbonic acid in the body which serves as the body's primary regulator of the acid/alkaline balance within the body. Therefore, if carbon dioxide is to be used to determine respiratory quotient, the steady state value of carbon dioxide production may have to be estimated from the decay profile of carbon dioxide production during the test, given the short test duration, or the test will have to be lengthened. Such simple accommodations can be made to the algorithm presented here.

Algorithm Step 3.

In order to obtain the RMR parameter from the previous two steps. A further formulation known as the Weir equation can be used. The Weir equation calculates RMR from Δ_{O2} and Δ_{CO2} (Δ_{CO2} is either measured or assumed to be equal to 0.8*(Δ_{O2}). The Weir Equation is as follows (where Δ_{O2} and Δ_{CO2} are assumed to be in ml/min):
RMR=[3.9*(Δ_{O2})+1.1*(Δ_{CO2})]*1.44 (30)

Other techniques can also be employed to convert these gas consumption variables to RMR, however, the Weir equation is the equation most commonly used and most widely accepted in indirect calorimetry systems.

### Section 2

Hardware

The algorithm described in the previous section was designed with two primary considerations in mind. First, the algorithm was designed to be fast and accurate. Second the algorithm was designed to lend itself to integration into a system consisting of simple hardware. For example, the algorithm only requires the flow of air into the system at one flow rate. Ancillary gases such as nitrogen or helium which are expensive and require safety oversight are not employed. Further, since only air is introduced to the system, compressed gas (which is burdensome with respect to the periodic refilling requirement) is not needed. Since only one flow of gas is needed, complex proportionate flow delivery systems with their drawbacks with respect to cost and dynamic performance limitations are unnecessary. A simple, accurate, and low cost sonic flow nozzle (a.k.a. critical flow venturi, or sonic choke) may be used for flow delivery. Such nozzles, coupled with appropriate upstream pressure and temperature sensing systems, have been known to outperform even mass flow controllers with respect to flow delivery accuracy and stability. In addition, the relatively slow change of oxygen levels within the chamber lends itself to the use of galvanic fuel cell oxygen sensors, which are cheap, and can be configured to produce astonishing resolution (such as the Sable Systems FC-10A fuel cell oxygen system, published resolution of 0.00001%). Further, when the cells are periodically re-zeroed, they can also produce astonishing short-term accuracy. The only two drawbacks of galvanic fuel cell oxygen sensors are their slow response (not a problem in this application) and the need to periodically replace them. Fortunately, galvanic fuel cells have steadily improved over the years and some modern fuel cells only require replacement at 24 month intervals, which would represent a reasonable period between factory calibration/service for any modern sensor-based system.

What follows is a common sense implementation of a hardware system tailored to the algorithm derived in Section 1 of this patent. The system's subcomponents can be divided in any number of ways, however, the author prefers to break them up into the following functional or conceptual groupings: 1. The chamber itself. 2. The pneumatic components. 3. The sensing systems. 4. The algorithm (already covered). Each of these topics will now be discussed in the order presented.

The Pod

The pod, shell or chamber is an enclosure in which the subject sits and is enclosed during testing. It is relatively small with respect to a typical human adult perhaps occupying 10 times or less the volumetric size of an adult. Business and marketing considerations make it preferable that the pod has a small footprint, perhaps 20 square feet or less, such that the pod can be placed in high foot traffic areas having limited available space such as in pharmacies, gyms, malls and shopping centers. FIG. 5 shows the preferred embodiment of the pod. Ideally the pod will be rounded and “egg-like” with an clam-shell rotating upper/frontal portion 32 as this configuration lends itself to easy mounting and dismounting by the subject. The upper/forward rotating clamshell might open either automatically at the end of a test or by request of the subject, or it might be a passive mechanical system similar to the hatchback damper/manual latch system of a car. It could also be closed by electromagnetic means. Regardless of the configuration, the upper/forward clamshell must be designed with (preferably redundant) safety release systems such that subjects can exit the pod manually, regardless of the state of the test being performed. The pod must have a method for two-way communication between the microprocessor system and the subject such as a flat panel display and keyboard 31. Possibilities include but are not limited to those shown in Table 2.

TABLE 2
Communication methodologies possible with the pod

| Communication | Communication |
|---|---|
| to Microprocessor | from Microprocessor |
| Voice Recognition | Voice Synthesis |
| Touch Pad | Flat Screen Display |
| Mouse Pad | CRT |
| Track Ball | LEDs |
| Keyboard | Projection Display |
| Touchscreen | OLED Display |
| Switch | Paper Printer |

One embodiment of the device contains not only a display inside the pod 31 as shown in the preferred embodiment, but also a display outside the pod 35 to “entice” potential users to take the test. Such enticement might also take the form of voice synthesis or celebrity voice or video recording outside the device and any number of other marketing oriented devices. The display device on the outside of the pod might also allow for data input in the case of an occupant who is incapable of interfacing with the system himself (e.g. an outside attendant could control the test). The preferred embodiment consists of a flat screen, form-fitting OLED, or projected heads-up display placed in front of the user to both guide the user through the test and to pitch advertisements for weight loss supplements, gyms etc. . . . This screen, in the preferred embodiment, would have a touch screen user interface in which the user answers questions from the microprocessor by touching the screen in various locations. The programming shown on this heads up display might again include appearances from celebrity's, advertisements, virtual (avatar) guides etc. . . . The pod must have a strong fan which circulates gas within the pod. This is necessary as the algorithm presented in section 1. assumes perfect mixing of the gases inside the chamber. The pod may or may not have a means of controlling humidity and carbon dioxide content. Dehumidification might improve the comfort level of the subject. Similarly, carbon dioxide (calcium/barium carbonate) scrubbers such as sodalime or baralime might be employed to reduce the discomfort associated with carbon dioxide rebreathing. Such scrubber agents would ideally be deployed in the airstream of the fan to optimize its scrubbing performance. The pod in its preferred embodiment has a window 32, either one-way or two-way, to allow the subject to look out of the device. Such a window would serve to reduce or eliminate discomfort and stress associated with confinement in the small space. The pod must have a (preferably comfortable) seat 31 for the user to sit in. The pod may rest on a load cell 33 as discussed earlier for weighing the subject. The pod includes, in the preferred embodiment, a payment accepting system to allow users to swipe a credit card or input cash as payment for a test or for diet or other services offered during testing. Finally, and perhaps most evident, the pod must be airtight in the closed configuration with the only gas flow into and out of the pod being controlled by the algorithm.

Other considerations in the design of the pod are a locking mechanism 36 to prevent users from occupying the device until a payment for the test has been proffered. In addition, PulMedics believes that the pod is the ideal platform in which to pitch different diet plans tailored to the metabolic requirements of the subject.

The Pneumatic Components:

The pneumatic components of the system are responsible for directing gas flow throughout the system. As mentioned earlier, the algorithm has been designed to minimize the complexity of the system. Indeed, the pneumatic schematic shows a relatively simple implementation of system plumbing (FIG. 3.). On the gas introduction side of the schematic, a compressor 3 is responsible for pressurizing incoming gas. This gas is then introduced into a heat exchanger 4 to cool the newly compressed gas to both increase patient comfort and to reduce the inlet-gas vs. pod-gas temperature gradient which might adversely effect the accuracy of the algorithm. A water trap 5 exists to trap condensate resulting from the compression and cooling of the gas. Next a pressure regulator 6 is introduced to maintain the pressure of the gas at a preset level. The pressure-regulated gas is then put through a restrictor 7/accumulator 8 section (a.k.a. pneumatic RC) to damp out pressure oscillations resulting from compressor operation. A overpressure-relief one-way valve 9 (a.k.a. popoff valve) is included in this pathway to ensure that the gas does not over-pressurize in the system. A restrictor based gas outlet path might also be included to ensure a steady state pressure can be achieved by the system by bleeding a small amount of gas overboard. The gas then passes an on/off 2-way/2-position solenoid valve 10 which can either allow gas to flow into the pod, or prevent gas from flowing into the pod. When flowing into the pod, the gas passes through a sonic flow nozzle 11. The sonic flow nozzle produces a steady, accurate flow level typically in the 1% to sub 1% accuracy range. On the gas release side of the pod, gas exiting the pod first passes though a filter 15 to protect the downstream sensors. A differential pressure screen element flow sensor 16 is used to determine gas flow. This sensor is preferably heated to prevent condensate from forming on the screen element. Alternately, other types of gas flow sensors may be used here. The gas passes through a second on/off 2-way/2 position solenoid valve 20 which can either allow gas to exit the pod or prevent gas from exiting the pod. An overpressure-relief one-way valve 17 is placed in this pneumatic pathway to prevent over-pressurization of the pod in the event of a flow system failure. Finally, the waste gas is dumped overboard (allowing it to escape to the atmosphere) at the outlet of the solenoid valve 21. This pneumatic schematic represents a simple system in which gas is either allowed to flow through the system at one flow rate, or is not allowed to flow though the system altogether. Alternate pneumatic designs may appear which perform the same or similar functions, indeed the potential number of combinations and permutations of various pneumatic technologies would be impossible to cover with reasonable brevity. Therefore, PulMedics claims this gas flow strategy and functional equivalents thereof, used for the purpose of assessing subject metabolic status, respiratory function, or subject body composition tests, as assessed by those skilled in the art, to be within the intellectual domain of this invention.

The Sensor Systems:

The author has decided to cover the sensor systems from a systems level or perspective. Specifically, electronic implementations of the sensor systems, such as amplification and analog filtering, are not provided here as such implementations should be reasonably obvious (see FIG. 4) to those skilled in the art. To echo earlier statements at the risk of being redundant, the system is designed for a minimalist implementation in hardware. The number of sensors used on this system is surprisingly low. On the gas inlet side, gas flow does not need to be sensed as the sonic flow nozzle produces a known, steady, and repeatable flow level. Sonic flow nozzles do require a pressure 22 and temperature 23 sensor to be placed upstream of the nozzle for correction in the microprocessor code of flow though the sonic flow nozzle for upstream pressure and temperature flow effects. Such sensors have been included in this system and are noted in the pneumatic schematic. It should be noted that all sensor systems include solenoid valves 12, 13, 14, 18, 19 which allow the sensors to sense from multiple locations in the system. Since the accuracy and precision of the sonic flow nozzle is a function of these upstream sensors, pressure and temperature sensors with a high degree of accuracy and resolution should be employed. It is also considered good form, when using a sonic flow nozzle, to include a humidity sensor 24 and, therefore, such a sensor is included in this system. On the gas exit side of the pod, a flow sensor 16,a temperature sensor 23, a humidity sensor 24, and an ambient pressure sensor 22 are included to allow the algorithm to correct the gas exiting the chamber to STPD conditions. The pressure sensor is a 30 PSIA (or higher) sensor which is configured with the solenoid valve to measure upstream gas pressure, downstream gas pressure, and ambient or barometric pressure.

All sensor technologies should be designed to maintain stable performance over time and factory calibrated against (preferably) primary standards. Finally, all sensor inputs to the microprocessor should be properly amplified 27, filtered 28 and sampled 29 to remove noise and aliasing artifact associated with digital sampling at the processor 30.

### BRIEF DESCRIPTION OF THE DRAWINGS

FIG. 1. Plot showing volumetric oxygen decay in pod over time in the two serially implemented flow regimes. The plot also indicates possible sampling locations for data to solve the equations in Steps 1 and 2.

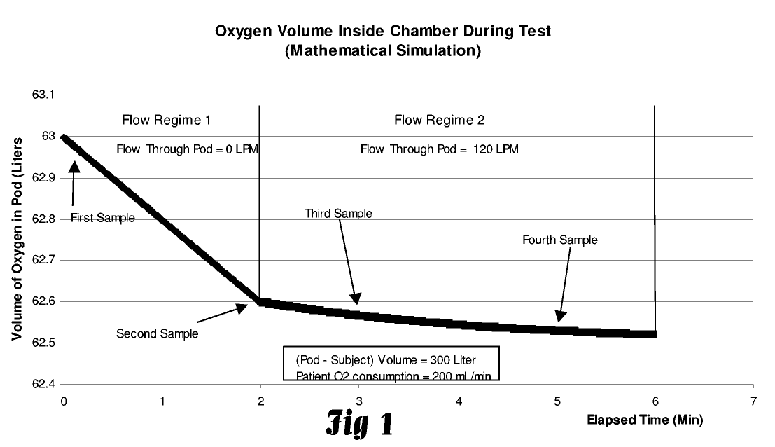

FIG. 2. Plot showing intersection of non-linear equations resulting from sampling at two time intervals using Equation 26. The expected solution of V=300 Liters is evident.

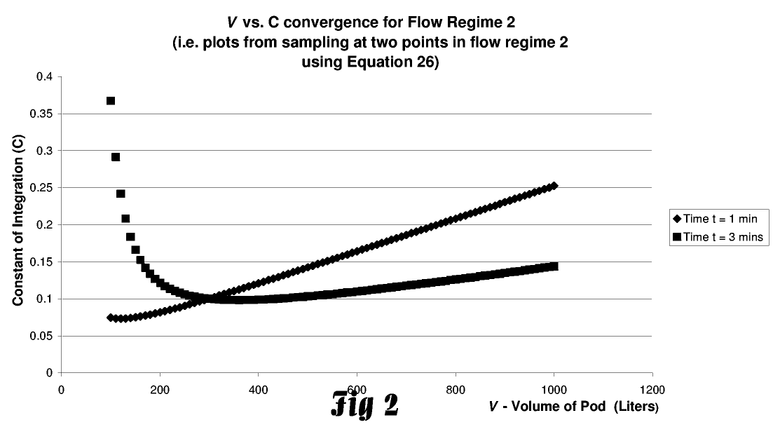

FIG. 3. Typical pneumatic schematic of system. The light circles represent sensor systems.

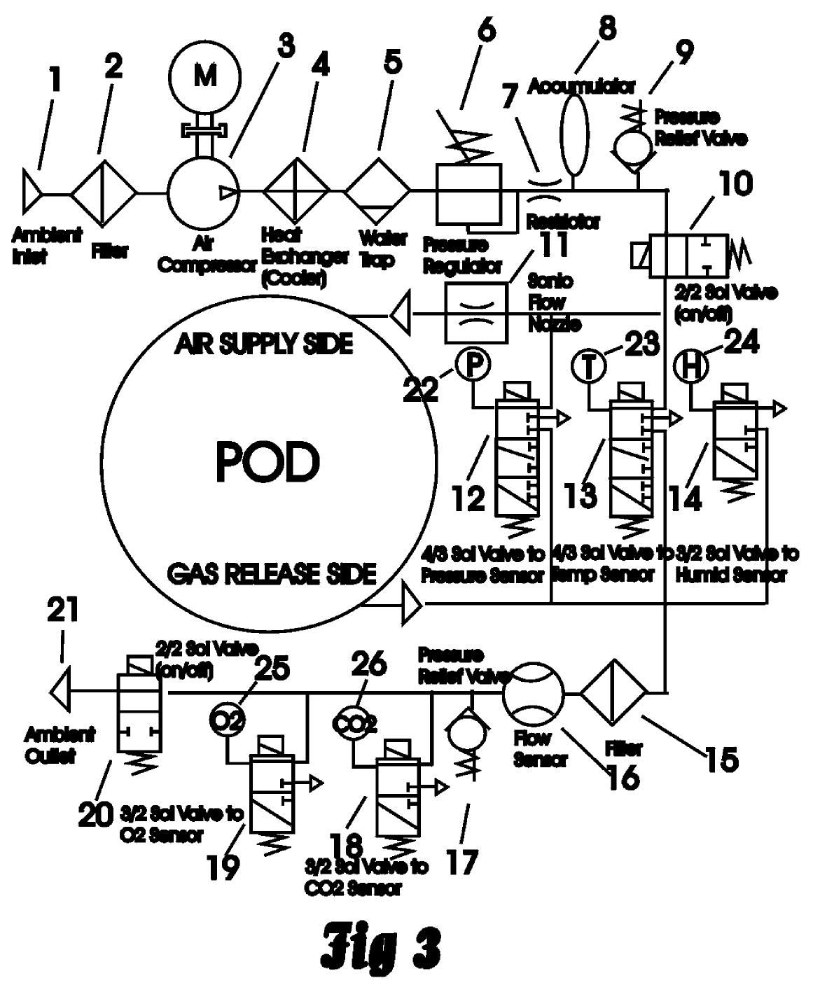

FIG. 4. Typical sensor sampling methodology.

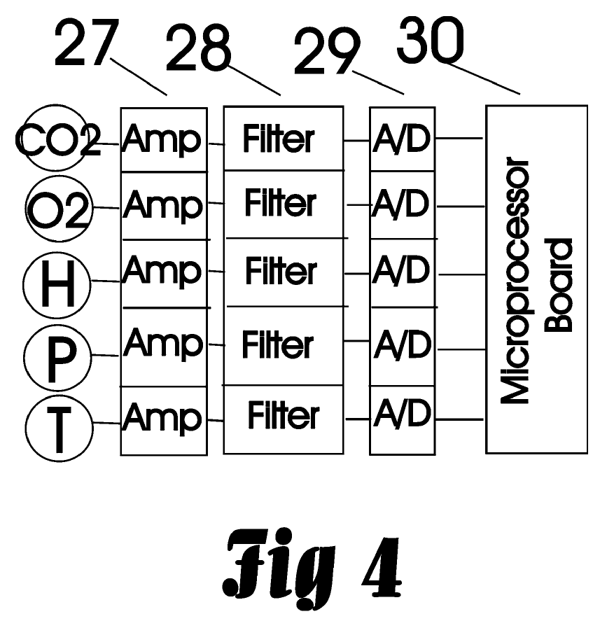

FIG. 5. General Overview of System.

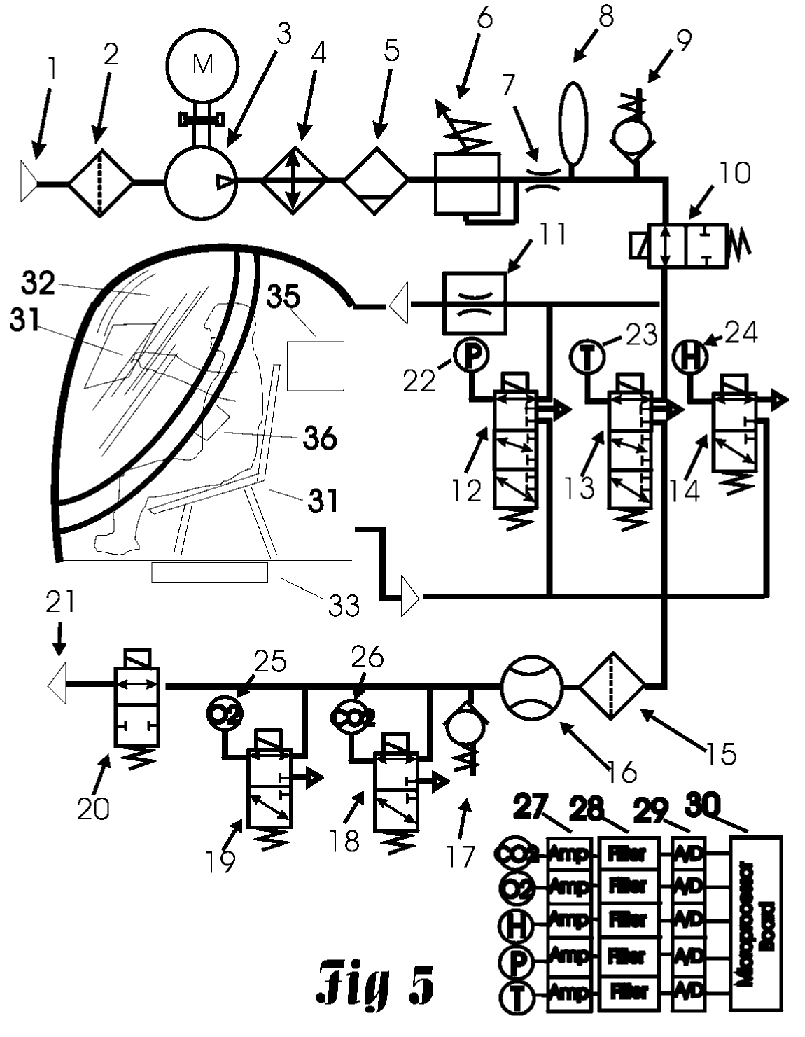

What is claimed is:

1. A whole-body indirect calorimeter for the determination of metabolic rate of a human subject, comprising:
means of presenting a chamber which encloses a subject to be measured;
means of measuring the metabolic rate of a subject within said chamber wherein said measurement of metabolic rate is preformed primarily by one or more gas partial pressure sensors, and wherein said measurement is completed in 60 minutes or less.

2. The whole-body indirect calorimeter of claim 1 wherein a flow inlet and flow outlet are provided and a source of gas flow through said inlet and said outlet is provided.

3. The whole-body indirect calorimeter of claim 1 wherein the oxygen consumption is measured by the use of a galvanic fuel cell oxygen sensor.

4. The whole-body indirect calorimeter of claim 1 wherein pressures within and outside said chamber are measured by a pressure transducer.

5. The whole-body indirect calorimeter of claim 1 wherein humidity within and outside said chamber are measured by a humidity sensor.

6. The whole-body indirect calorimeter of claim 1 wherein temperature within and outside said chamber are measured by a temperature sensor.

7. The whole-body indirect calorimeter of claim 1 further comprising a means of determining the respiratory quotient through the use of a carbon dioxide sensor.

8. The whole-body indirect calorimeter of claim 1 further comprising a means of determining percent body fat through the use of a load cell.

9. A whole-body indirect calorimeter for the determination of the metabolic rate of a human subject, comprising:
means of presenting a chamber which encloses a subject to be measured wherein said chamber occupies less than 5000 liters;
means of using the change in partial pressure of one or more breathing gases over time to determine the resting metabolic rate of a subject occupying said chamber.

10. The whole-body indirect calorimeter of claim 9 wherein the metabolic rate is measured primarily by the use of a galvanic fuel cell oxygen sensor.

11. The whole-body indirect calorimeter of claim 9 wherein pressures within and outside said chamber are measured by a pressure transducer.

12. The whole-body indirect calorimeter of claim 9 wherein humidity within and outside said chamber are measured by a humidity sensor.

13. The whole-body indirect calorimeter of claim 9 wherein temperature within and outside said chamber are measured by a temperature sensor.

14. The whole-body indirect calorimeter of claim 9 further comprising a means of determining the respiratory quotient through the use of a carbon dioxide sensor.

15. The whole-body indirect calorimeter of claim 9 further comprising a means of determining percent body fat through the use of a load cell.

16. A whole-body indirect calorimeter for the determination of the metabolic rate of a human subject, comprising:
means of presenting a chamber which encloses a subject to be measured;
means of using the change in partial pressures of gases within said chamber to determine the volume of the subject occupying said chamber;
means of using said determination of subject volume as a precursor in the calculation of the metabolic rate of the subject occupying said chamber.

17. The whole-body indirect calorimeter of claim 16 wherein said metabolic rate is measured primarily by the use of a galvanic fuel cell oxygen sensor.

18. The whole-body indirect calorimeter of claim 16 wherein pressures within and outside said chamber are measured by a pressure transducer.

19. The whole-body indirect calorimeter of claim 16 wherein humidity within and outside said chamber are measured by a humidity sensor.

20. The whole-body indirect calorimeter of claim 16 wherein temperature within and outside said chamber are measured by a temperature sensor.

21. The whole-body indirect calorimeter of claim 16 further comprising a means of determining the respiratory quotient through the use of a carbon dioxide sensor.

22. The whole-body indirect calorimeter of claim 16 further comprising a means of determining percent body fat through the use of a load cell.
